(19) United States
(12) Patent Application Publication
(10) Pub. No.: US 2015/0330928 A1
(43) Pub. Date: Nov. 19, 2015

(54) DEVICE FOR DETERMINING A SALT CONCENTRATION OF CRUDE OIL

(71) Applicant: Bartec ORB, Chicago, IL (US)

(72) Inventor: Douglas Korslund, Riverside, IL (US)

(73) Assignee: Bartec ORB, Chicago, IL (US)

(21) Appl. No.: 14/280,075

(22) Filed: May 16, 2014

(51) Int. Cl. G01N 27/07; G01N 33/28

(52) U.S. Cl. CPC G01N 27/07; G01N 33/2835

(57) ABSTRACT

A device for determining a salt concentration of crude oil includes a mixture container for holding a mixture of crude oil and solvent while the salt concentration is determined. The device also may include a sample conduit possessing a first end selectively fluidly connected to one of a crude oil storage container and a solvent pump and a second end selectively fluidly connected to one of a crude oil pump and the mixture container. In addition, the device may include an air pump configured to supply a sample conduit with pressurized air, and at least one collapsible solvent storage container configured to collapse when solvent is withdrawn from the solvent storage container.

### FIELD OF DISCLOSURE

The present disclosure relates to a device for determining a salt concentration of crude oil and, more particularly, to a device that automates the process of measuring the salt concentration of a crude oil sample in an offline environment.

### BACKGROUND

A relatively common practice in the refinement and production crude oil is to measure the salt concentration of the crude oil. Knowledge of the salt concentration is helpful in deciding whether the crude oil should be desalted. Crude oil with an excessive salt concentration tends to increase corrosion of refining equipment and interfere with catalysts employed in the refining process.

One test for determining the salt concentration of crude oil is set forth in ASTM D3230, the contents of which are hereby incorporated by reference. This test involves measuring the electrical conductivity of crude oil due to the presence of chlorides such as sodium chloride, calcium chloride and magnesium chloride. The electrical resistivity of crude oil is generally too high for conventional sensors to accurately measure electrical conductivity. The crude oil is therefore mixed with solvents such as methanol, butanol and xylene to reduce its electrical resistivity. These solvents tend to be relatively flammable and volatile.

The test outlined in ASTM D3230 can be implemented in an online environment or an offline environment. Performing the test online involves connecting a measurement apparatus to a pipeline carrying crude oil through a refinery or other industrial facility. An example of such an online measurement apparatus is the “Model P-600 Salt in Crude Analyzer” sold by Orb Instruments, Inc. The solvents employed in the ASTM D3230 test are typically stored on site. Because refineries and industrial facilities oftentimes experience elevated temperatures, there is an increased risk of fire or explosion resulting from storing the solvents on site. To reduce this risk, the solvents are usually stored in bulky, rigid containers, which can be relatively expensive to manufacture and install.

Offline testing involves measuring a collected sample of crude oil in a non-industrial setting, such as a laboratory, where the flammability and volatility of the solvents are less of a concern. Generally, a laboratory technician mixes the crude oil sample and the solvents in a beaker, inserts a probe into the beaker to obtain an electrical conductivity measurement, and then reads a salt concentration from a chart based on the electrical conductivity measurement. Precise amounts of the crude oil must be mixed, in a specific sequence, by the laboratory technician to obtain an accurate electrical conductivity measurement. Frequent calibrations of the probe may also be necessary. In some cases, the probe may have to be calibrated before each measurement. Offline testing therefore tends to be time-consuming and susceptible to human error due to improper mixing.

### SUMMARY

A device for determining a salt concentration of crude oil that includes a sample conduit possessing first and second ends, a crude oil pump, a solvent pump and a mixture container. The crude oil pump is selectively fluidly connected to one of a crude oil storage container and the second end of the sample conduit. The solvent pump is configured to withdraw solvent from a solvent storage container. The mixture container is configured to hold a mixture of the crude oil and the solvent while the salt concentration of the crude oil is determined. The first end of the sample conduit is selectively fluidly connected to one of the crude oil storage container and the solvent pump. The second end of the sample conduit is selectively fluidly connected to one of the crude oil pump and the mixture container.

In one case, the device includes a solvent storage container for storing a solvent, a solvent pump, a sample conduit possessing first and second ends, a mixture container, and an air pump. The solvent pump is configured to withdraw the solvent from the solvent storage container. The mixture container is configured to hold a mixture of crude oil and the solvent while the salt concentration of the crude oil is determined. The first end of the sample conduit is selectively fluidly connected to one of the solvent pump and a crude oil supply. The second end of the sample conduit is selectively fluidly connected to the mixture container. The air pump is configured to supply the first end of the sample conduit with pressurized air.

In one case, the device includes a collapsible solvent storage container, a solvent pump, a mixture container and a sample conduit. The collapsible solvent storage container includes a sealed interior for storing a solvent and is configured to collapse when the solvent is withdrawn from the sealed interior. The solvent pump is configured to withdraw the solvent from the sealed interior of the collapsible solvent storage container. The mixture container is configured to hold a mixture of crude oil and the solvent while the salt concentration of the crude oil is determined. A first end of the sample conduit is selectively fluidly connected to one of the solvent pump and a crude oil supply. A second end of the sample conduit is selectively fluidly connected to the mixture container.

### BRIEF DESCRIPTION OF THE DRAWINGS

FIG. 1 is a schematic hydraulic circuit of a measurement device in accordance with principles of a first example of the present disclosure;

FIG. 2 illustrates the control system of the measurement device shown in FIG. 1;

FIG. 3 illustrates an example of a screen displayed by an external computer of the measurement device;

FIG. 4 depicts a perspective view of an exterior of the measurement device illustrated in FIG. 1;

FIG. 5 illustrates a schematic hydraulic circuit of a measurement device in accordance with principles of a second example of the present disclosure; and

FIG. 6 depicts a schematic hydraulic circuit of a measurement device in accordance with principles of a third example of the present disclosure.

### DETAILED DESCRIPTION

Generally speaking, a measurement device according to the present disclosure is able to automatically measure a salt concentration of a crude oil sample in an offline environment such as a laboratory. The measurement device includes a crude oil distribution system that automatically measures a predetermined volume of crude oil and distributes the crude oil to an electrical conductivity detection system where the crude oil is mixed together with a predetermined volume of solvent, measured and supplied by a solvent distribution system, to lower the electrical resistivity of the crude oil. After the electrical conductivity detection system is supplied with the crude oil and solvents, a pressurized air system supplies the electrical conductivity detection system with pressurized air to mix the crude oil and the solvents. The electrical conductivity detection system measures the electrical conductivity of the resulting mixture, and a control system determines the salt concentration of the crude oil by referencing a table, created during an earlier calibration process, of corresponding salt concentration and electrical conductivity values. The control system automatically controls the crude oil distribution system, the solvent distribution system, the pressurized air system and the electrical conductivity detection system and thus alleviates an operator from having to manually measure and mix the crude oil and solvents to determine the salt concentration.

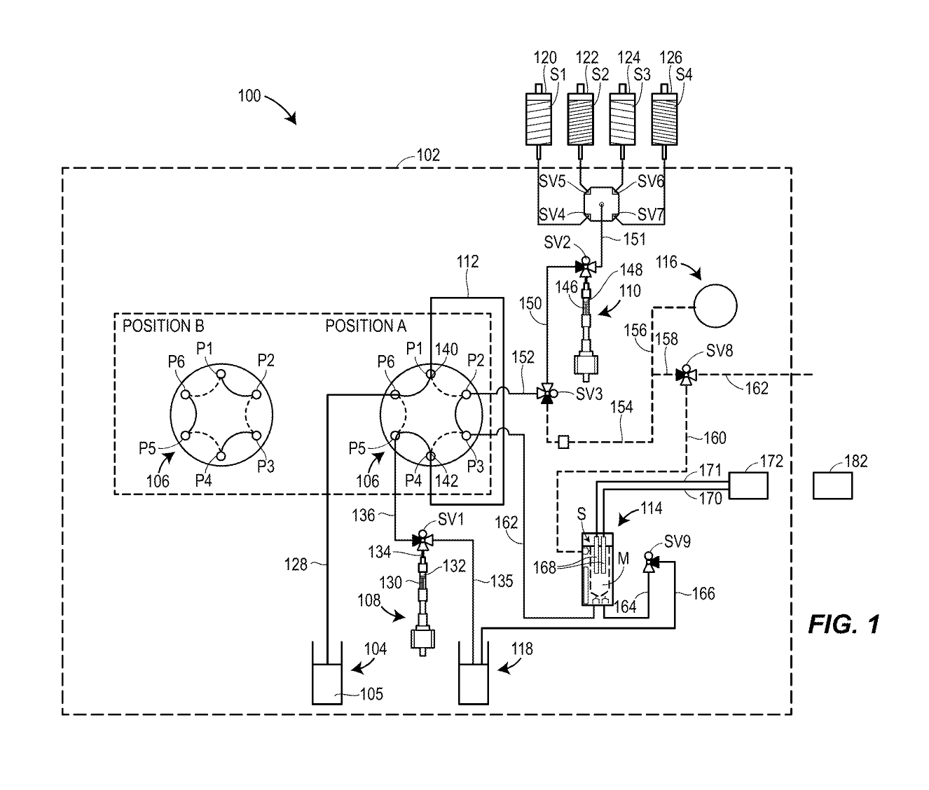

FIG. 1 illustrates an example measurement device 100 including a crude oil distribution system and a solvent distribution system that function to supply an electrical conductivity detection system with a mixture of crude oil and solvents. The crude oil distribution system includes a crude oil storage container 104 that holds the crude oil 105 and a crude oil pump 108 configured to withdraw a predetermined volume of the crude oil 105 from the crude oil storage container 104 and subsequently discharge the withdrawn crude oil 105 to the electrical conductivity detection system. The solvent distribution system includes a solvent pump 110 operable to withdraw solvents S1, S2, S3 and S4 individually from a plurality of storage containers 120, 122, 124 and 126 located exterior to a housing 102 of the measurement device 100 and then discharge the withdrawn solvents S1, S2, S3 and S4 to the electrical conductivity detection system where at least some of the solvents S1, S2, S3 and S4 are mixed together with the crude oil 105 in a mixture container 114. In one embodiment, the crude oil and solvent distribution systems share a selector valve 106 and a sample conduit 112 for delivering the crude oil 105 and the solvents S1, S2, S3 and S4 to the mixture container 114. To promote mixing of the crude oil 105 and the solvents S1, S2, S3 and S4 in the mixture container 114, the measurement device 100 includes a pressurized air system that supplies the mixture container 114 with pressurized air from an air pump 116 to generate a bubbling effect in the mixture container 114. The air pump 116 is also configured to drive fluid out of the mixture container 114 and into a waste container 118 upon completion of the salt concentration measurement.

Generally during operation of the measurement device 100, the crude oil pump 108 withdraws crude oil 105 from the crude oil storage container 104 and injects the withdrawn crude oil 105 into the sample conduit 112 such that the sample conduit 112 is filled with a predetermined volume of the crude oil 105. The solvent pump 110 is used to withdraw the solvents individually from the solvent storage containers 120, 122, 124, and 126 and push them into the sample conduit 112. The air pump 116 supplies pressurized air to expel the solvents and the crude oil 105 from the sample conduit 112 and into mixture container 114, and also to create bubbles in the mixture M in the mixture container 114. The electrical conductivity of the mixture M held in the mixture container 114 is then measured, and compared to a lookup chart, to determine the salt concentration of the crude oil 105.

So configured, the measurement device 100 automates the process of determining the salt concentration of a crude oil sample. In one embodiment, the measurement device 100 is configured to perform the salt concentration test outlined in ASTM D3230. The measurement device 100 frees an operator or laboratory technician from having to perform the relatively time-consuming steps of manually mixing the solvents and the crude oil, and manually calibrating an electrical conductivity probe. The measurement device 100 also reduces the likelihood that improper amounts of the solvents and crude oil are mixed as the result of human error. Additionally, as described further below, the measurement device 100 does not require a pressurized source of crude oil and therefore can be implemented in an offline environment. One benefit of operating the measurement device 100 in an offline environment is that there is a reduced risk of fire and explosion. The solvent storage containers therefore do not require a bulky and/or rigid design, and in some cases, as described further below, can be configured in a replaceable and/or disposable manner.

Each of the foregoing components of the measurement device 100, and the methods of operating the measurement device 100, are described below in more detail.

The crude oil distribution system includes the crude oil storage container 104 which stores the crude oil 105 prior to the salt concentration measurement. In one embodiment, the crude oil storage container 104 is a removable container such as beaker. The crude oil storage container 104 may be enclosed entirely within the housing 102 as depicted in FIG. 1 or may be outside the housing 102. The crude oil storage container 104 may be filled with the crude oil 105 by pouring the crude oil 105 into an open end of the crude oil storage container 104 so that the crude oil storage container 104 stores the crude oil 105 in an un-pressurized manner. The housing 102 may include a retractable cover or door, as described further below, providing access to the crude oil storage container 104. The volume of the crude oil storage container 104 may be large enough to provide crude oil for several salt concentration measurements. A conduit 128 fluidly connects the crude oil storage container 104 to the selector valve 106.

The crude oil distribution system withdraws the crude oil 150 from the crude oil storage container 104 by way of the crude oil pump 108. After withdrawing the crude oil 105, the crude oil pump 108 injects the crude oil into the sample conduit 112. As illustrated in FIG. 1, the crude oil pump 108 is a syringe including a plunger 130 slidably disposed within a chamber 132. Movement of the plunger 130 in one direction creates suction that draws the crude oil 105 into the chamber 132, and movement of the plunger 130 in the opposite direction creates pressure that expels the crude oil 105 from the chamber 132. A controllable electric motor EM1, described below in more detail, actuates the plunger 130. The crude oil pump 108 is not limited to a syringe-type arrangement and can be any suitable pump including a gear pump.

The crude oil distribution system fluidly connects the crude oil pump 108 to the selector valve 106 by way of conduits 134 and 136 and a solenoid valve SV1. The solenoid valve SV1 may be configured as a 3-way valve with a normally-closed port connected to the conduit 134 and a normally-open port connected to a conduit 135 leading to the waste container 118.

The ability of the crude oil pump 108 to withdraw the crude oil 105 from the crude oil storage container 104 and inject the withdrawn crude oil 105 into the sample conduit 112 makes it unnecessary to connect the measurement device 100 to a pressurized source of crude oil such as a refinery pipeline. The measurement device 100 is therefore suitable for operation in offline environments including laboratories or other testing facilities.

The sample conduit 112 includes a first end 140 and a second end 142 which are each in fluid communication with the selector valve 106. An interior passageway extends between the first and second ends 140 and 142 of the sample conduit 112 and, in some embodiments, possesses a volume equal to or substantially equal to the volume of the crude oil sample needed to perform a salt concentration measurement, for example, according to the method outlined in ASTM D3230. In one case, the volume of the interior passageway of the sample conduit 112 is equal to approximately (e.g., ±10%) 10 mL, or lesser or greater. By filling the sample conduit 112 with the crude oil 105, the crude oil distribution system obtains a sample of the crude oil 105 having a predetermined volume.

Turning to the solvent distribution system, the solvent pump 110 is configured to withdraw the solvents S1, S2, S3 and S4 from the solvent storage containers 120, 122, 124 and 126, and inject the withdrawn solvents S1, S2, S3 and S4 into the sample conduit 112. The solvent pump 110 illustrated in FIG. 1 is configured as a syringe with a plunger 146 slidably disposed within a chamber 148. Movement of the plunger 146 in one direction creates suction that draws a solvent into the chamber 148, and movement of the plunger 146 in the opposite direction creates pressure that expels the withdrawn solvent from the chamber 148. A controllable electrical motor EM2, which is further described below, actuates the plunger 146. As an alternative to the syringe-type arrangement, the solvent pump 110 may be a gear pump, or any other type of pump capable of moving solvent from the solvent storage containers 120, 122, 124 and 126 to the sample conduit 112.

The solvent distribution system connects the solvent pump 110 to the selector valve 106 by way of solenoid valves SV2 and SV3 and conduits 150 and 152. The solenoid valve SV2 may be configured as a 3-way valve with a normally-closed port connected to the conduit 150 and a normally-open port connected to a conduit 151 leading to the solvent storage containers 120, 122, 124 and 126. The solenoid valve SV3 may be a 3-way valve having a normally-closed port connected to a conduit 154 leading to the air pump 116.

The solvent distribution system stores each of the solvents S1, S2, S3 and S4 in a different one of the solvent storage containers 120, 122, 124 and 126 prior to the salt concentration measurement. The solvents S1, S2, S3 and S4 are used to lower the electrical resistivity of the crude oil 105 so that electrical conductivity of the crude oil is easier to measure, and also, clean the conduits and other components of the measurement device 100 upon completion of the salt concentration measurement. In one embodiment, the solvents S1, S2, S3 and S4 are naphtha, butanol, methanol and xylene, respectively.

The solvent distribution system connects each of the solvent storage containers 120, 122, 124 and 126 to the conduit 151 by way of a respective one of the solenoid valves SV4, SV5, SV6 and SV7. The solenoid valves SV4, SV5, SV6 and SV7 are selectively openable to permit the solvent pump 110 to individually withdraw the solvents from the solvent storage containers 120, 122, 124 and 126 one at a time.

The solvent storage containers 120, 122, 124 and 126 are removably attached to an exterior of the housing 102. This feature makes it relatively easy to replace the solvent storage containers 120, 122, 124 and 126 when they become empty. In one embodiment, the solvent storage containers 120, 122, 124 and 126 are made of a flexible material, such as plastic, so that the solvent storage containers 120, 122, 124 and 126 collapse under the force of atmospheric pressure as the solvent is withdrawn from each of the solvent storage containers 120, 122, 124 and 126. The solvent storage containers 120, 122, 124 and 126 each possess a sealed interior that is filled with one of the solvents. As the solvent is withdrawn from the sealed interior, atmospheric pressure outside the solvent storage container causes the solvent storage container to collapse. In one embodiment, each of the solvent storage containers 120, 122, 124 and 126 is a plastic bag with sealable port. When one of the solvent storage containers 120, 122, 124 and 126 becomes empty, it can be replaced with a new solvent storage container. In this way, the solvent storage containers 120, 122, 124 and 126 are replaceable and/or disposable. Permanent, bulky and/or rigid metal solvent storage containers are not required because the measurement device 100 is implemented in an offline environment where the solvent storage containers are not likely to be exposed to harsh operating conditions. One advantage of including replaceable solvent storage containers is that the solvent storage containers can be pre-filled with the solvent so that the operator is not required to handle the solvents, which are oftentimes toxic.

While the solvent storage containers 120, 122, 124 and 126 of the present embodiment are arranged outside of the housing 102, alternative embodiments can be arranged differently, for example, with one or more of the solvent storage containers 120, 122, 124 and 126 positioned inside the housing 102.

The pressurized air system employs the air pump 116, powered by a controllable electric motor EM3, to supply pressurized air to the sample conduit 112 as well as the mixture container 114, via a plurality of conduits and valves. The air pump 116 is selectively fluidly connected to the first end 140 of the sample conduit 112, via the conduits 152, 154 and 156, the solenoid valve SV3 and the selector valve 106. By pumping pressurized air into the first end 140 of the sample conduit, the air pump 116 pushes fluid in the sample conduit 112 out of the second end 142 of the sample conduit 112 and into the mixture container 114 by way of the conduit 162. To pump pressurized air into an upper portion the mixture container 114, the pressurized air system utilizes conduits 156, 158 and 160 and a solenoid valve SV8 to selectively fluidly connect the air pump 116 to the mixture container 114. The solenoid valve SV8 may be configured as a 3-way valve with a normally-closed port connected to a conduit 158, and a normally-open port connected to a conduit 162 that vents to the exterior of the housing 102. Pumping pressurized air into the upper portion of the mixture container 114 with the air pump 116 is useful to discharge the mixture of crude oil and solvents from the mixture container 114 at the end of a salt concentration measurement as described in more detail below.

In one embodiment, the crude oil distribution system, the solvent distribution system and the pressurized air system each employ the selector valve 106 to selectively deliver the crude oil 105, the solvents 120, 122, 124 and 126 and the pressurized air to mixture container 114. The selector valve 106, which includes a plurality of ports P1-P6, is movable between a position A and a position B. The measurement device 100 illustrated in FIG. 1 arranges the selector valve 106 in position A. While FIG. 1 depicts the position B of the selector valve 106, this is done for explanatory purposes only. When the selector valve 106 is arranged in the position A, the selector valve 106 establishes fluid communication between the following pairs of ports: ports P1 and P6; ports P5 and P4; and ports P2 and P3. When the selector valve 106 is arranged in position B, the selector valve 106 establishes fluid communication between the following pairs of ports: ports P1 and P2; ports P3 and P4; and ports P5 and P6. The selector valve 106 may be configured to rotate between positions A and B, and a controllable electric motor EM4 may be included to rotate the selector valve 106.

Turning to the electrical conductivity detection system, this portion of the measurement device 100 includes the mixture container 114 in which the mixture M, composed of the crude coil 105 and at least some of the solvents S1, S2, S3 and S4, is held while a sensor S measures the electrical conductivity of the mixture M. The mixture container 114 is selectively fluidly connected to the second end 142 of the sample conduit 112 by way of the selector valve 106 and a conduit 162, as well as, selectively fluidly connected to the air pump 116 via the conduits 156, 158 and 160 and the solenoid valve SV8. In addition, the mixture container 114 is selectively fluidly connected to the waste container 118 by way of conduits 164 and 166 and a solenoid valve SV9. The solenoid valve SV9 may be a 3-way valve with a normally-open port in fluid communication with the conduit 164 and a normally-closed port in fluid communication with the conduit 166.

The sensor S possesses a pair of spaced apart electrodes 168 (e.g., platinum electrodes) at least partially submerged in the mixture M. Electrical lines 170 and 171 connect each of the electrodes 168 to an internal computer 172. Measuring the electrical conductivity of the mixture M involves supplying one of the electrodes 168 with a known voltage to induce a current in the other one of the electrodes 168. The internal computer 172 and/or an external computer 182 analyzes the electric current, or other electrical characteristic, to determine the electrical conductivity of the mixture M. Subsequently, the internal computer 172 and/or an external computer 182 cross-references the electrical conductivity of the mixture M with a lookup table to determine the salt concentration of the sample of crude oil, as described below in more detail. To increase the accuracy of the electrical conductivity measurement, the internal computer 172 and/or the external computer 182 may take into account the temperature of the mixture M by utilizing a temperature sensor (not illustrated) arranged inside the mixture container 114.

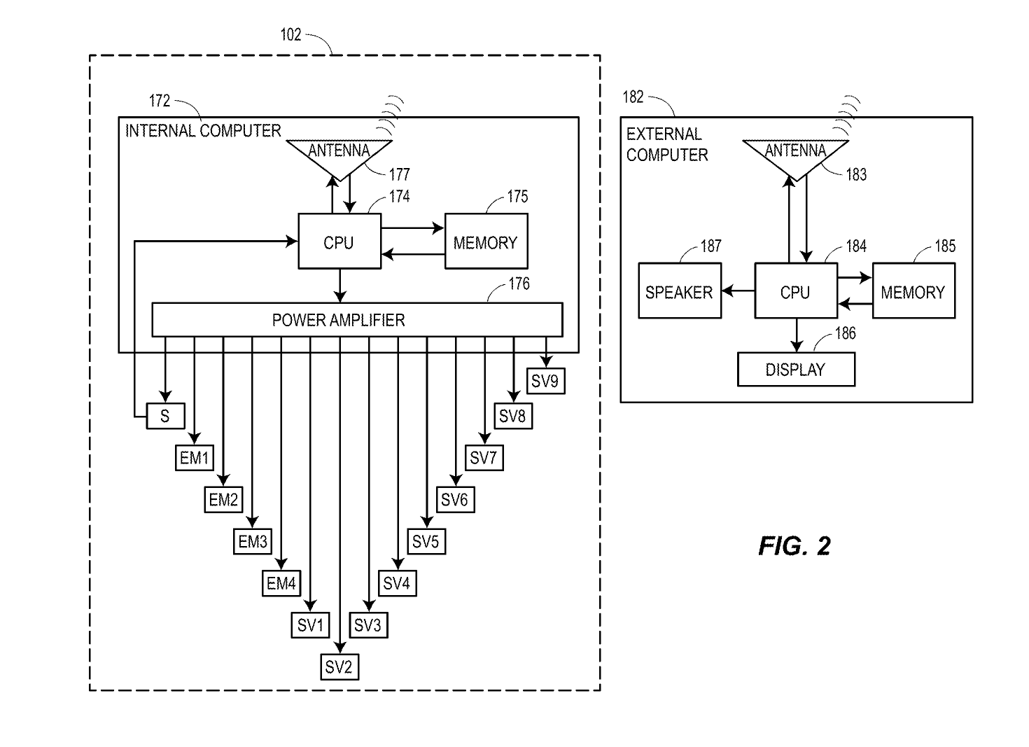

The control system, which includes the internal computer 172 and the external computer 182, controls the crude oil and solvent distribution systems, the pressurized air system and the electrical conductivity detection system. As illustrated in FIG. 2, the internal computer 172 is located inside the housing 102 of the measurement device 100 and includes a central processing unit (CPU) 174, a memory 175, a power amplifier 176 and an antenna 177. The memory 175 stores a program including instructions executable by the CPU 174. The CPU 174 executes the instructions and outputs control signals for controlling the sensor S, the electric motors EM1-EM4 and the solenoid valves SV1-SV9. The power amplifier 176 increases the power of the control signals so that the control signals are sufficient to power the sensor S, the electric motors EM1-EM4 and the solenoid valves SV1-SV9. After receiving a measurement signal from the sensor S representing the electrical conductivity of the mixture M, the CPU 174 generates an electrical conductivity value based on the measurement signal and wirelessly transmits the electrical conductivity value, via the antenna 177, to the external computer 182. The antenna 177 also wirelessly receives data and/or instructions from the external computer 182 for controlling the sensor S, the electric motors EM1-EM4 and the solenoid valves SV1-SV9.

The external computer 182 is located outside the housing 102 and may be a portable computer such as tablet computer (e.g., an iPad) or a smartphone (e.g., an iPhone). The external computer 182 wirelessly communicates with the internal computer 172 via an antenna 183. In one embodiment, the internal computer 172 and the external computer 182 employ their respective antennas 177 and 183 to wirelessly communicate with each other via a Bluetooth communication protocol. In addition to the antenna 183, the external computer 182 includes a CPU 184, a memory 185, a display 186 and a speaker 187. The memory 185 stores a program including a set of instructions executable by the CPU 184. The memory 185 may also store a lookup table, generated during an earlier calibration phase (described below in more detail), that correlates a plurality electrical conductivity values with a plurality of salt concentration values. Based on the electrical conductivity data received from the internal computer 172 via the antenna 183, the CPU 184 uses the lookup table to determine the salt concentration that corresponds to the electrical conductivity measured by the sensor S. The CPU 184 displays the salt concentration on the display 186, which may be a touchscreen interface including an LED panel.

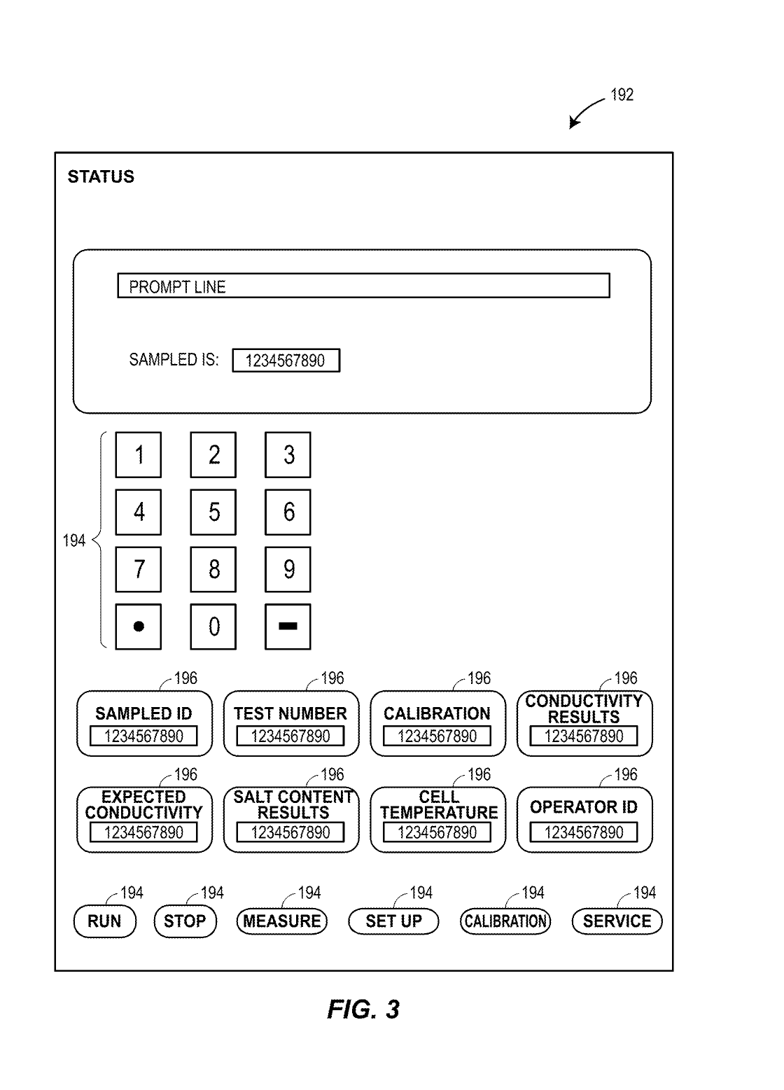

By using a touchscreen for the display 186, an operator can input data and instructions into the external computer 182 by selecting buttons displayed on the display 186. FIG. 3 illustrates an example of screen 192 for the display 186 which includes buttons 194 manually operable by an operator. For example, the “RUN” and “STOP” buttons allow the operator to start and stop an ongoing operation of the measurement device 100. The “MEASURE” button commences the salt concentration measurement of a crude oil sample. The “SET UP” button causes the display of another screen that allows the operator to input identification information and/or define parameters of the salt concentration measurement such as the volume of the crude oil sample. Selecting the “CALIBRATION” button causes CPU 184 to calibrate the sensor S by generating a lookup table of corresponding conductivity values and salt concentration valves, as described below. In addition to the buttons 194, the display screen 300 includes data fields 196 displaying information related to the current measurement. Some of the data fields 196 may be selectable. For example, selecting the “SALT CONTENT RESULTS” may result in the display of another screen including a graph charting the salt concentrations of multiple crude oil samples.

A method of determining the salt concentration of the crude oil 105 with the measurement device 100 is described below. Initially, the crude oil storage container 104 is filled with the crude oil 105. The crude oil storage container 104 is then placed inside the housing 102 with the conduit 128 submerged in the crude oil 105. In one embodiment, the crude oil storage container 104 may be filled with the crude oil 105 while the crude oil storage container 104 is positioned inside the housing 102.

Next, the selector valve 106 and the crude oil pump 108 are rinsed with the crude oil 105. This step involves controlling the electric motor EM4 to move the selector valve 106 to the position A so that the first end 140 of the fluid conduit 112 is fluidly connected to the crude oil storage container 104, and so that the second end 142 of the fluid conduit 112 is fluidly connected to the crude oil pump 108. The solenoid valve SV1 is energized to open the normally-closed port connected to the conduit 136. Next, the CPU 174 controls the electric motor EM1 in a manner causing the crude oil pump 108 to withdraw the crude oil 105 from the crude oil storage container 104 by way of the sample conduit 112. In the case where the crude oil pump 108 is configured as a syringe, this action is accomplished controlling the electric motor EM1 to move the plunger 130 away from the conduit 134. The chamber 132 of the syringe is consequently filled with the crude oil 105. Subsequently, the solenoid valve SV1 is de-energized so that the normally-closed port is closed. The plunger 130 is then moved upwardly to expel the crude oil 105 from the chamber 132 through the conduit 135 and into the waste container 118. The above-described rinsing process may be repeated a second time to further rinse to selector valve 106 and crude oil pump 108 with the crude oil 105.

Following the rinsing process, the sample conduit 112 is filled with a predetermined volume of the crude oil 105. This step involves controlling the electric motor EM4 to move the selector valve 106 to the position B so that fluid communication is established between the ports P5 and P6. The solenoid valve SV1 is energized to open the normally-closed port connected to the conduit 136. The plunger 130 of the crude oil pump 108 is moved downwards to withdraw the crude oil 105 from the crude oil storage container 104 and thereby fill the chamber 132. Subsequently, the selector valve 106 is moved to the position A, thereby fluidly connecting the crude oil pump 108 and the sample conduit 112. The plunger 130 is then moved in the upward direction to eject the crude oil 105 from the chamber 132 and into the sample conduit 112. Filling the sample conduit 112 with the crude oil 105 creates a sample of the crude oil 105 having a predetermined volume.

Xylene is then added to the sample conduit 112 by opening the solenoid valve SV7 and withdrawing a proper volume of xylene from the solvent storage container 126 with the solvent pump 110 by activating the electric motor EM2. The solvent storage container 126 collapses as the xylene is withdrawn. If the solvent pump 110 is configured as a syringe, this is accomplished by pulling down on the plunger 146 to fill the chamber 148 with the proper volume of xylene. The volume of xylene withdrawn from the solvent storage container 126 may be determined by the test set forth in ASTM D3230, or any other suitable test.

Subsequently, the solenoid valve SV5 is energized to open the normally-closed port connected to the conduit 150. The plunger 146 is then moved upwards to expel the xylene from the chamber 148 and into the first end 140 of the sample conduit 112. This action pushes some of the crude oil 105 out of the second end 142 of the sample conduit 112 and into the conduit 162 leading to the mixture container 114.

Next, an alcohol mixture of solvents is added to the sample conduit 112. The solenoid valve SV2 is de-energized so that the normally-closed port returns to the closed position. The solenoid valve SV5 is opened, and a proper volume of the butanol is withdrawn from the solvent storage container 122 by the solvent pump 110. The solvent storage container 122 collapses as the butanol is withdrawn. The solenoid valve SV5 is then closed and the solenoid valve SV6 is opened so that the solvent pump 110 can withdraw a proper volume of the methanol from the solvent storage container 124. The solvent storage container 124 collapses as the methanol is withdrawn. The volume of butanol and methanol withdrawn from the solvent storage containers may be determined by the test set forth in ASTM D3230, or any other suitable test.

Next, the normally-closed port of the solenoid valve SV2 is opened, and the plunger 146 is moved upwards to expel the alcohol mixture containing the methanol and the butanol from the chamber 148 and into the first end 140 of the sample conduit 112. This action ejects an additional amount of the crude oil 105 from the second end 142 of the sample conduit 112 and into the conduit 162.

To push any residual fluid in the sample conduit 112 into the mixture container 114, the solenoid valve SV3 is energized to open the normally-closed port connected to the conduit 154, and the electric motor EM3 of the air pump 116 is turned on. The air pump 116 introduces pressurized air into the first end 140 of the sample conduit 112 and expels residual fluid in the sample conduit 112 into the mixture container 114. Thereafter, the selector valve 106 is moved to the position A so that the air pump 116 pushes any remaining fluid in the conduit 162 into the mixture container 114. To encourage mixing of the fluids in the mixture container 114, the air pump 116 supplies pressurized air to the mixture container 114 for a predetermined amount of time. The pressurized air creates bubbles in the mixture container 114 which enhance mixing of the crude oil 105 and the solvents. When the fluids are sufficiently mixed, the solenoid valve SV3 is de-energized and the electric motor EM3 of the air pump 116 is turned off to allow the mixture M to stabilize. Any pressurized air remaining in the mixture container 114 is vented to the exterior of the housing 102 via the conduits 160 and 162.

Measuring the electrical conductivity of the mixture M in the mixture container 114 involves the following steps. The CPU 174 and the power amplifier 176 supply one of the electrodes 168 with a known voltage via the electric line 170. As a result, the mixture M conducts an electric current between the electrodes 168, and an electric signal is transmitted, via the electric line 171, to the CPU 174 of the internal computer 172. Subsequently, the CPU 174 determines the electrical conductivity of the mixture M based on the electric signal from the sensor S, and then wirelessly transmits this information, via the antenna 177, to the external computer 182. In an alternative embodiment, the electrical conductivity determination is performed by the CPU 184.

Next, the external computer 182 determines the salt concentration of the crude oil 105 based on the electrical conductivity information received from the internal computer 172. The CPU 184 of the external computer 182 receives the electrical conductivity information through the antenna 182 and subsequently compares the electrical conductivity information to the lookup table stored in the memory 185. The lookup table, which is populated during an earlier calibration of the sensor S (described below in more detail), correlates a plurality of electrical conductivity values with a plurality of salt concentration values. The CPU 184 finds the salt concentration value in the lookup table that corresponds to the electrical conductivity value measured by the sensor S. The CPU 184 subsequently displays the salt concentration value on the display 186 and stores the salt concentration value in the memory 185.

While the external computer 182 of the present embodiment ultimately determines the salt concentration by referencing the lookup table stored in the memory 185, in other embodiments, the salt concentration determination may be performed solely by the internal computer 172 and thereafter transmitted to the external computer 182 for display.

After the salt concentration has been determined, the mixture M is ejected from the mixture container M into the waste container 118 by opening the normally-closed port of the solenoid valve SV7, opening the normally-closed port of the solenoid valve SV9 and turning on the air pump 116. Upon completion of the ejection process, the normally-closed ports of the solenoid valves SV7 and SV8 are closed and the air pump 116 is turned off.

The mixture container 114 is then cleaned with naphtha from the solvent storage container 120 by opening the solenoid valve SV4 and withdrawing naphtha from the solvent storage container 120 with the solvent pump 110. The solenoid valve SV5 is energized to open the normally-closed port and the withdrawn naphtha is pushed by the solvent pump 110 through the conduits 150, 152 and 162 and into the mixture container 116. These steps are repeated to add another load of naphtha to the mixture container 114. The solenoid valves SV4 and SV5 are then turned off, and the solenoid valve SV3 is turned on to open the normally-closed port connected to the conduit 154. The air pump 116 is subsequently turned on to supply pressurized air to the mixture container 114 to circulate the naphtha in the mixture container 114 and thereby clean the mixture container 114. The solenoid valve SV3 is turned off and the solenoid valves SV7 and SV8 are turned on so that the naphtha is drained to the waste container 118 via the conduits 164 and 166. Finally, the solenoid valves SV7 and SV8 and the air pump 116 are turned off.

Prior to the salt concentration determination described above, the measurement device 100 may perform a calibration sequence to create a lookup table that correlates a plurality of electrically conductivity values with a plurality of salt concentration values. The calibration process involves using the measurement device 100 to measure the electric conductivities of multiple crude oil samples, each having a known salt concentration. The electrical conductivity measurements of each of the known crude oil samples is performed in a manner similar that described above, including mixing each of the known crude oil samples, separately, with the solvents. The lookup table is populated with the measured electrical conductivity values and the known salt concentration values (which may be previsouly manually inputted by the operator). The memory 175 of the internal computer 172 and/or the memory 185 of the external computer 182 stores the lookup table so that, later, when the crude oil sample of a unknown salt concentration is to be determined, the lookup table can be used to find a salt concentration value corresponding to the measured electrical conductivity. The foregoing calibration sequence, with the exception of the input of the known salt concentration values, is performed entirely by the measurement device 100. Therefore, the operator is freed from having to manually perform each and every step of the calibration process.

In one embodiment, the mixture container 114 is configured to accommodate different types of sensors S for measuring the electrical conductivity of the mixture M. For instance, in addition to the 2-electrode sensor S illustrated in FIG. 1, the mixture container 114 may be able to accommodate a 4-electrode sensor S. To keep track of which type of sensor S is currently connected to the mixture container 114, the measurement device 114 may include sensor detector. Different types of sensors S typically have different mounting plates, of different sizes, for connecting the sensor S to the mixture container 114. In one embodiment, the sensor detector determines the type of sensor S connected to the mixture container 114 by detecting the size of the mounting plate of the sensor S. The sensor detector includes a button switch that is depressed when a large mounting plate is used, and which is not depressed when a small mounting plate is used. The type of sensor S detected by the sensor detector is displayed on the display 186 so that the operator knows the type of sensor S currently being used by the measurement device 100 and does not have to look inside the measurement device 100.

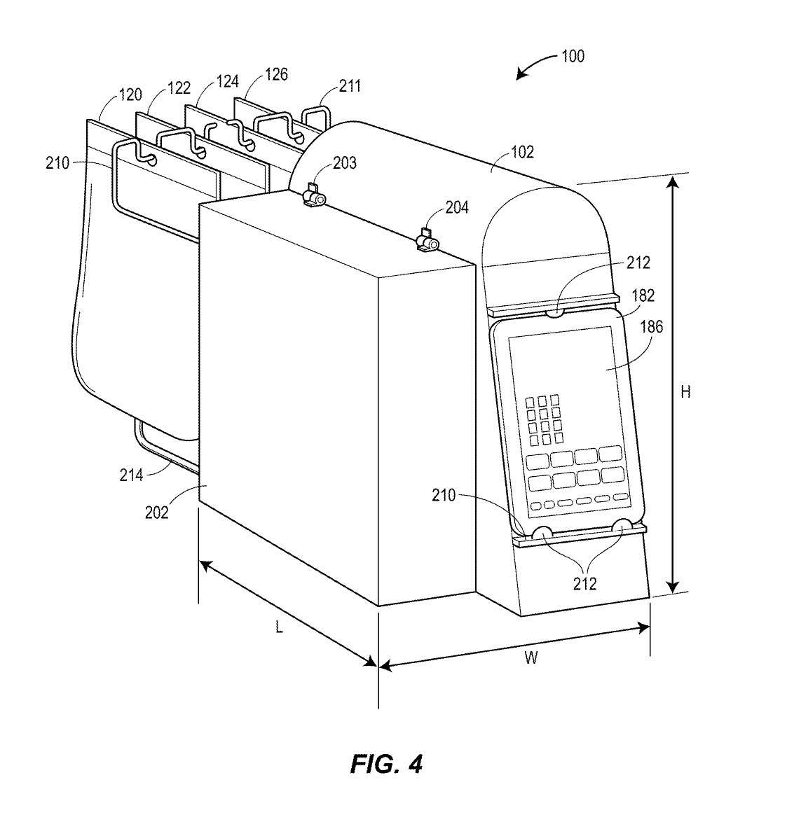

FIG. 4 depicts a perspective view of the exterior of the measurement device 100. In one embodiment the housing 102 possesses a height H of approximately (e.g., ±10%) 14 inches, a length L of approximately (e.g., ±10%) 17 inches and a width of approximately (e.g., ±10%) 9.5 inches. The housing 102 includes a door 202 rotatably connected to the rest of the housing 102 by a pair of hinges 203, 204. Opening the door 202 provides access to the interior of the housing 102 so that, for example, the crude oil storage container 104 can be re-filled.

The solvent storage containers 120 and 122 are hung on a bracket 210 attached to the rear face of the measurement device 100. The solvent storage containers 124 and 126 are hung on a bracket 211 attached to the rear face of the measurement device 100. Hanging the solvent storage containers 120, 122, 124 and 126 in this manner makes it relatively easy to replace the solvent storage containers 120, 122, 124 and 126 when they become empty. The solvent storage containers 120, 122, 124 and 126 depicted in FIG. 2 are configured as sealed plastic bags. Each of the plastic bags is fluidly connected to a port (not depicted) on the rear face of the housing 102 by a respective plastic tube 214.

The external computer 182 is removably mounted to the exterior of the measurement device 100. FIG. 4 shows that the external computer 182 rests on an outwardly protruding ledge 210 of the housing 102 and slides under lip members 212 so that the external computer 182 does not fall forward. This setup is advantageous if the external computer 182 is a relatively thin portable computer, such as an iPad, which can be easily slid under the lip members 212.

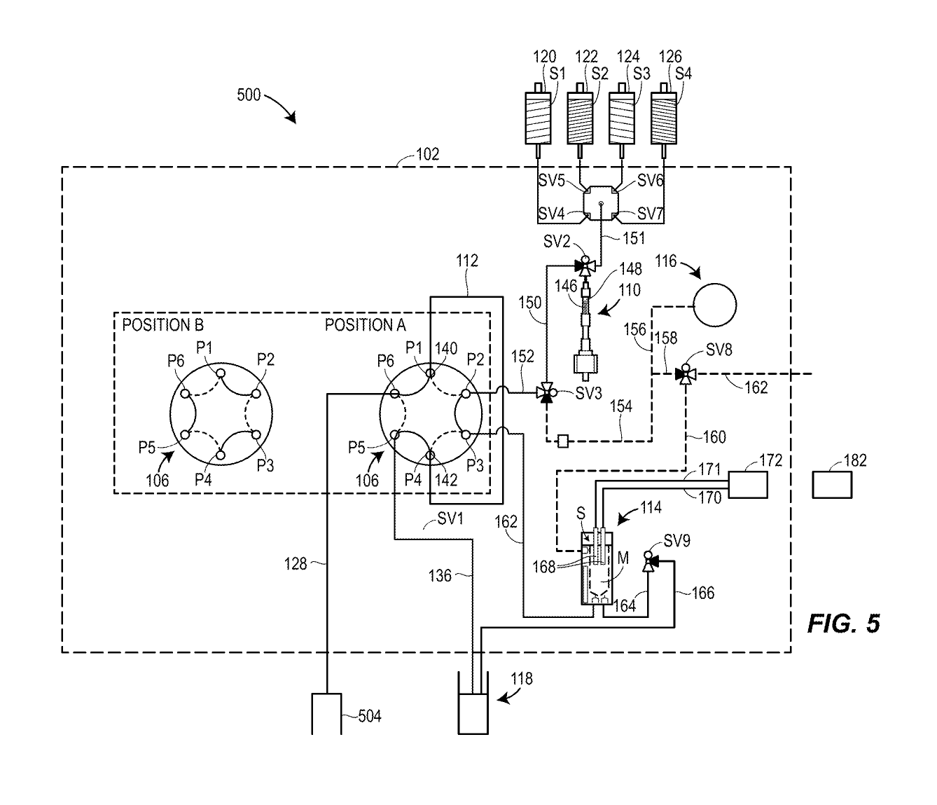

While the foregoing embodiment of the measurement device includes a crude oil storage container and a crude oil pump, in other embodiments, the device is free of such components. The measurement device 500 illustrated in FIG. 5 is similar to the measurement device 100 depicted in FIG. 1, except that the device 500 lacks a crude oil storage container and a crude oil storage pump. The measurement device 500 is supplied with crude oil from an external pressurized supply of crude oil 504. The pressurized supply of crude oil 504 may be a crude oil pipeline. The device 500 is therefore suitable for us in an online setting such as an oil refinery. Another way in which the measurement device 500 differs from the measurement device 100 is that the waste container 118 is positioned exterior to the housing 102.

Operation of the measurement device 500 is similar to the measurement device 100, except that the sample conduit 112 is not filled with crude oil by way of a crude oil storage pump. The sample conduit 112 is filled with crude oil by fluidly connecting the first end 140 of the sample conduit 112 to the pressurized supply of crude oil 504. The pressurized crude oil flows under its own power into the sample conduit 112.

Similar to the measurement device 100, the measurement device 500 includes an air pump 116 for supplying pressurized air to move fluid from the sample conduit 112 to the mixture container 114, and to drain fluid from the mixture container 114 upon completion of the salt concentration measurement. The inclusion of the air pump 116 makes the device 500 suitable for use in an environment where an external supply of pressurized air is not readily available.

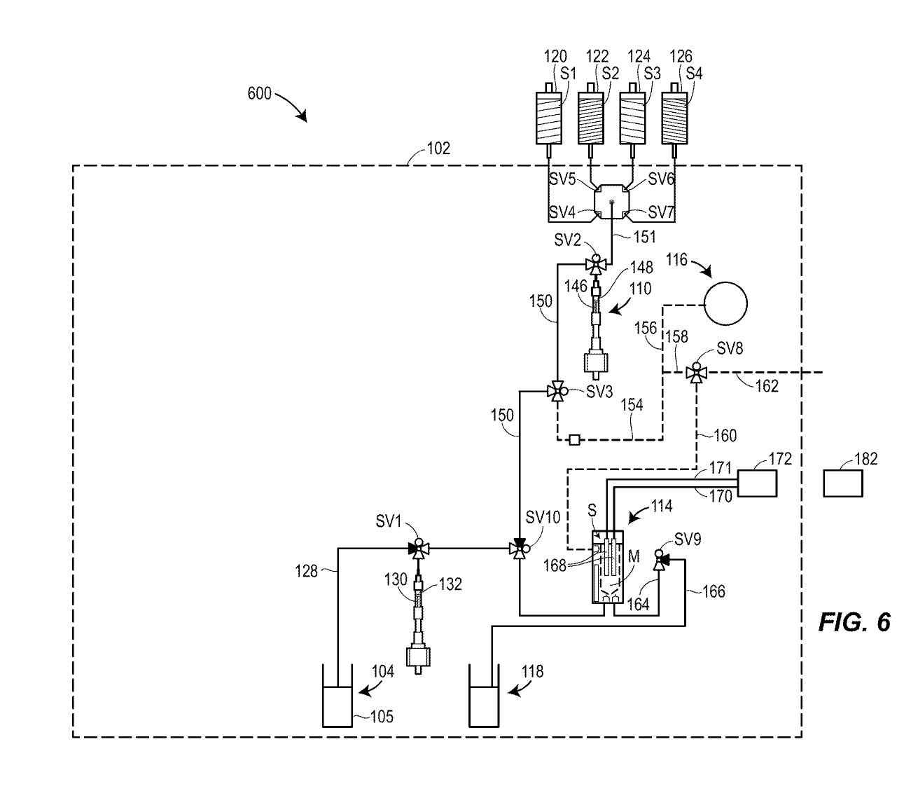

Each of the abovementioned embodiments employs a selector valve to selectively fluidly connect the crude oil and solvent distribution systems to the electrical conductivity detection system, and also, a sample conduit to measure a crude oil sample with a predetermined volume. Alternative embodiments of the measurement device, such as the one illustrated in FIG. 6, may not include the selector valve and the sample conduit. The measurement device 600 illustrated in FIG. 6 is similar in many respects to the measurement device 100 depicted in FIG. 1, but differs from the measurement device 100 in that the measurement device 600 is free of the selector valve 106 and the sample conduit 112. Instead, the measurement device 600 employs the crude oil pump 108, which is configured as a syringe, to measure a sample of the crude oil 105 with a predetermined volume. The crude oil pump 108 then expels the crude oil 105 directly to the mixture container 114 via conduits 602 and 604 and a selector valve SV10. The selector valve SV10 may be configured as a 3-way valve with a normally-open port connected to the conduit 602 and a normally-closed portion connected to the conduit 150 leading to the solvent pump 110 and the air pump 116.

The operation of the measurement device 600 is similar to the measurement device 100, except that the crude oil 105 and the solvents S1, S2, S3 and S4 are not pumped through a selector valve or a sample conduit. When it is time to inject the mixture container 114 with the sample of crude oil 105, the internal computer 172 controls the solenoid valve SV10 to fluidly couple the conduit 602 and the conduit 602, thereby allowing the crude oil pump 108 to directly inject the crude oil sample into the mixture container 114. When it is time to inject the mixture container 114 with one of the solvents S1, S2, S3 and S4 or pressurized air, the internal computer 172 controls the solenoid valve SV10 to fluidly couple the conduit 150 and the conduit 604, so that the solvent pump 110 and the air pump 116 can directly inject the solvents or pressurized air into the mixture container 114. The omission of the selector valve and the sample conduit helps reduce costs and complexity of the measurement device.

While the foregoing embodiments have been described in a manner in which the electrical conductivity of the mixture of crude oil and solvents is measured, the scope of the present disclosure is not limited to this configuration. Rather, alternative embodiments of the device could be arranged and configured to measure a different electrical characteristic of the mixture such as the electrical resistivity of the mixture.

While the present disclosure has been described with respect to certain embodiments, it will be understood that variations may be made thereto that are still within the scope of the appended claims.

What is claimed is:

1. A device for determining a salt concentration of crude oil comprising:
a sample conduit possessing a first end and a second end;
a crude oil pump selectively fluidly connected to one of a crude oil storage container and the second end of the sample conduit;
a solvent pump configured to withdraw solvent from a solvent storage container;
a mixture container for holding a mixture of the crude oil and the solvent while the salt concentration of the crude oil is determined; and
the first end of the sample conduit being selectively fluidly connected to one of the crude oil storage container and the solvent pump, and the second end of the sample conduit being selectively fluidly connected to one of the crude oil pump and the mixture container.

2. The device of claim 1, the crude oil pump being energizable to: (i) withdraw the crude oil from the crude oil storage container when the crude oil pump is fluidly connected to the crude oil storage container, and (ii) inject the withdrawn crude oil into the sample conduit when the crude oil pump is fluidly connected to the second end of the sample conduit to fill the sample conduit with a predetermined volume of the crude oil.

3. The device of claim 1, wherein the crude oil pump is a syringe including a plunger slidably disposed within a chamber.

4. The device of claim 1, comprising the solvent storage container.

5. The device of claim 4, the solvent storage container including a sealed interior for storing the solvent, the solvent storage container being configured to collapse during withdrawal of the solvent from the sealed interior by the solvent pump.

6. The device of claim 5, comprising:
the crude oil storage container; and
a housing enclosing the crude oil storage container, the crude oil pump and the mixture container, wherein the solvent storage container is removably attached to an exterior of the housing.

7. The device of claim 1, comprising a sensor for determining the salt concentration of the crude oil, the sensor including at least two electrodes positioned inside the mixture container.

8. The device of claim 1, comprising a selector valve movable between a first position and a second position, the selector valve fluidly connecting the second end of the sample conduit and the crude oil pump when the selector valve is arranged in the first position, the selector valve fluidly connecting the second end of the sample conduit and the mixture container when the selector valve is arranged in the second position.

9. The device of claim 8, the selector valve fluidly connecting the first end of the sample conduit and the crude oil storage container when the selector valve is arranged in the first position, the selector valve fluidly connecting the first end of the sample conduit and solvent pump when the selector valve is arranged in the second position.

10. The device of claim 1, comprising an air pump configured to supply the first end of the sample conduit with pressurized air.

11. A device for determining a salt concentration of crude oil comprising:
a solvent storage container for storing a solvent;
a solvent pump configured to withdraw the solvent from the solvent storage container;
a mixture container for holding a mixture of crude oil and the solvent while the salt concentration of the crude oil is determined;
a sample conduit possessing a first end and a second end, the first end being selectively fluidly connected to one of the solvent pump and a crude oil supply, the second end being selectively fluidly connected to the mixture container; and
an air pump configured to supply the first end of the sample conduit with pressurized air.

12. The device of claim 11, comprising a housing enclosing the mixture container and the sample conduit, wherein the solvent storage container is removably attached to an exterior of the housing.

13. The device of claim 12, the solvent storage container including a sealed interior for storing the solvent, the solvent storage container being configured to collapse during withdrawal of the solvent from the sealed interior by the solvent pump.

14. The device of claim 11, comprising a selector valve movable between a first position and a second position, the selector valve fluidly connecting the first end of the sample conduit the crude oil supply when the selector valve is arranged in the first position, the selector valve fluidly connecting the second end of the sample conduit and the mixture container when the selector valve is arranged in the second position.

15. A device for determining a salt concentration of crude oil comprising:
a collapsible solvent storage container including a sealed interior for storing a solvent, the collapsible solvent storage container being configured to collapse when the solvent is withdrawn from the sealed interior;
a solvent pump configured to withdraw the solvent from the sealed interior of the collapsible solvent storage container;
a mixture container configured to hold a mixture of crude oil and the solvent while the salt concentration of the crude oil is determined; and
a sample conduit possessing a first end and a second end, the first end being selectively fluidly connected to one of the solvent pump and a crude oil supply, the second end being selectively fluidly connected to the mixture container.

16. The device of claim 15, comprising a housing enclosing the mixture container and the sample conduit, wherein the solvent storage container is removably attached to an exterior of the housing.

17. The device of claim 15, wherein the solvent pump is a syringe including a plunger slidably disposed within a chamber.

18. The device of claim 15, comprising a selector valve movable between a first position and a second position, the selector valve fluidly connecting the first end of the sample conduit and the crude oil supply when the selector valve is arranged in the first position, the selector valve fluidly connecting the second end of the sample conduit and the mixture container when the selector valve is arranged in the second position.

19. The device of claim 18, the selector valve fluidly connecting the solvent pump and the first end of the sample conduit when the selector valve is arranged in the second position.

20. The device of claim 15, comprising a sensor for determining the salt concentration of the crude oil, the sensor including at least two electrodes positioned inside the mixture container.

21. A device for determining a salt concentration of crude oil comprising:
a crude oil syringe configured to withdraw a predetermined volume of crude oil from a crude oil storage container;
a solvent syringe configured to withdraw a predetermined volume of solvent from a solvent storage container;
a mixture container for holding a mixture of the crude oil and the solvent while the salt concentration of the crude oil is determined; and
the crude oil syringe being selectively fluidly connected to one of the crude oil storage container and the mixture container, the solvent syringe being selectively fluidly connected to one of the solvent storage container and the mixture container.

22. The device of claim 21, comprising the crude oil storage container and the solvent storage container.

23. The device of claim 22, comprising a housing enclosing the crude oil syringe, the solvent syringe and the mixture container, wherein the solvent storage container is removably attached to an exterior of the housing.
